(19) United States
(12) Patent Application Publication
(10) Pub. No.: US 2008/0183130 A1
(43) Pub. Date: Jul. 31, 2008

(54) Balloon Catheter

(76) Inventor: Georg Lutter, Kiel (DE)

Correspondence Address: POPOVICH, WILES & O'CONNELL, PA;650 THIRD AVENUE SOUTH, SUITE 600, MINNEAPOLIS, MN, 55402, US

(21) Appl. No.: 10/592,838

(22) PCT Filed: Mar. 11, 2005
(86) PCT No.: PCT/DE05/00437

§ 371 Date: Oct. 31, 2006

(30) Foreign Application Priority Data
Mar. 16, 2004 (DE) 10 2004 013 012.4

(51) Int. Cl. A61M 25/10

(52) U.S. Cl. 604/96.01

(57) ABSTRACT

The invention relates to a balloon catheter (1) for the sealing of blood vessels and cardiac chambers with at least one inflatable chamber (20) connected to a first line (10), characterized by a unit (40) adjacent to the at least one inflatable chamber (20), forming a cavity with said chamber (20) into which a vacuum line (30) opens, whereby separator elements (60, 70) are provided between the walls forming the cavity, which is gas-permeable at least in a limited region (50) on the outside thereof, for sucking the balloon catheter (1) to the blood vessel or the cardiac chamber.

This invention concerns a balloon catheter which will be used to seal areas of diseased blood vessels or cardiac chambers. Especially, this patent refers to a balloon catheter which is applied for minimally invasive procedures in human hearts.

The correction of a heart valve disease is the most frequently performed operation in heart surgery. Normally open heart valve procedures are done under cardiac arrest and direct bloodless view. Parallel to these operations one can also undertake minimally invasive procedures on a beating heart (closed chest operations). Therefore, special applicable tools are necessary to reach the operating field by passing through the cardiovascular system.

When performing this kind of operation special perfusion catheters, as for instance this balloon catheter (DE 195 33 601), are going to be used. The U.S. Pat. No. 6,135,981, for instance, proposes a perfusion catheter with two distal adjacent inflatable chambers which create a separate operating space. This operating space will be excluded from the blood circulatory system. In addition, the surface of these occluding balloons (like U.S. Pat. No. 5,423,745) can be designed with special superficial structures, as local twisted or circular convexities or protrusions.

The patent DE 102 17 559 describes an equipment with two inflatable dilatation units alongside a catheter for the ablation of insufficient or stenotic heart valves. The dilatation units are specially arranged: the distal dilatation unit will be subvalvular and the proximally located dilatation unit will be above the aortic valve. This device enables a fluid-sealed closure with the wall of the vessel and creates an inner bloodfree working area in which the surgeon will be able to treat the aortic valve with special catheter tools under direct view.

The proximal toroidal-formed dilatation unit is able to perform an optimal closure with the ascending aorta. In contrast, the positioning of the distally-placed toroidal-formed dilatation unit can cause leaking due to the anatomic circumstances.

Therefore, the aim of this invention is to construct a catheter which allows a guaranteed and controllable sealing of cardiovascular areas.

This problem will be solved by a balloon catheter for the sealing of blood vessels and cardiac chambers: this catheter with at least one inflatable chamber connected to a first line, characterised by a unit adjacent to the at least one inflatable chamber, forming a cavity with said chamber into which a vacuum line opens, whereby separator elements are provided between the walls forming the cavity, which is gas permeable at least in a limited region on the outside therefore, for sucking the balloon catheter to the blood vessel or the cardiac chamber.

This catheter of the invention can draw in the environmental tissue due to applying a vacuum at the cavity which consists of the unit and the chamber wall. Out of this function the described invention has the advantage of getting a form-fitted sealing of the balloon-catheter with its environment in situ.

The invention will be illustrated by the following figures:

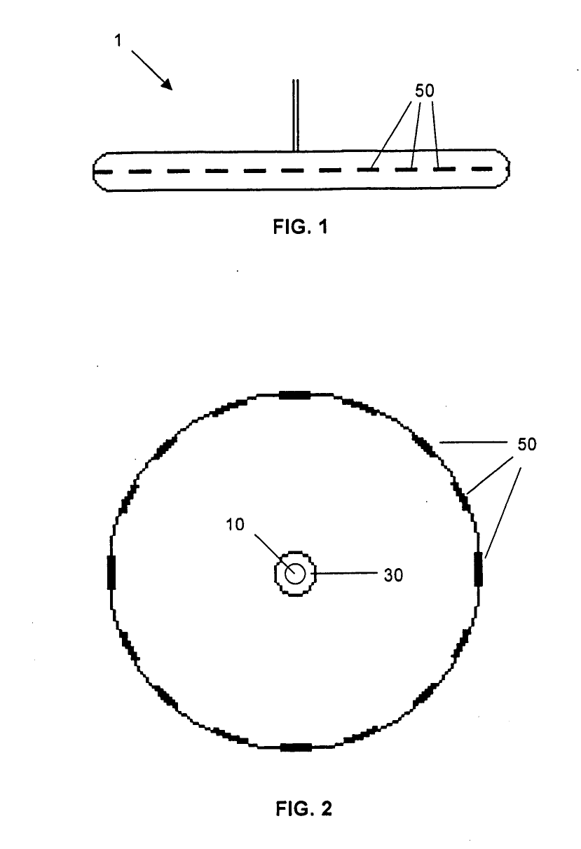

FIG. 1 Lateral view according to the balloon catheter of the invention;

FIG. 2 View from above of the balloon catheter of FIG. 1;

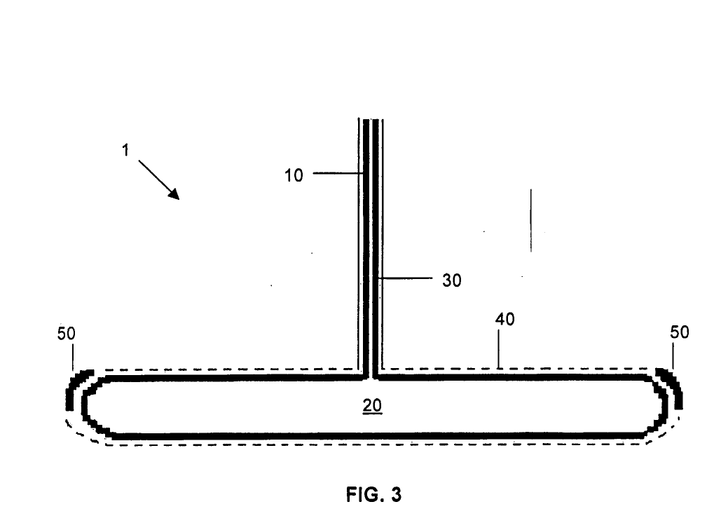

FIG. 3 Cross section of a lateral view of a preferred example of this invention;

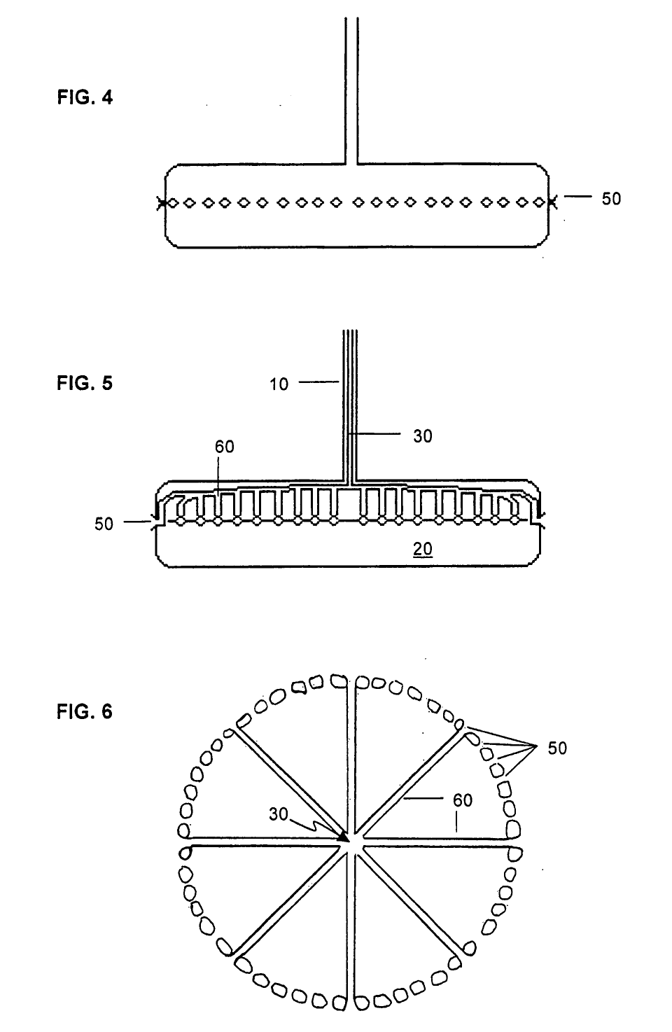

FIG. 4 Lateral view of a preferred example of this invention;

FIG. 5 Cross section of a lateral view of the balloon catheter of FIG. 4;

FIG. 6 Cross section of a top view of the balloon catheter of FIG. 4;

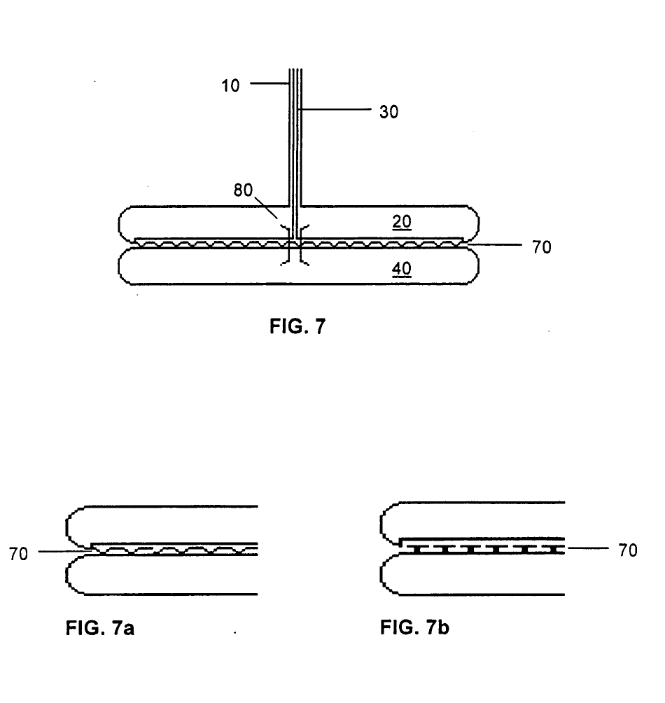

FIG. 7 Lateral view of a preferred example of this invention;

FIG. 7a Enlargement of separator elements of FIG. 7

FIG. 7b Enlargement of separator elements as an alternative to the separator elements illustrated in FIG. 7a;

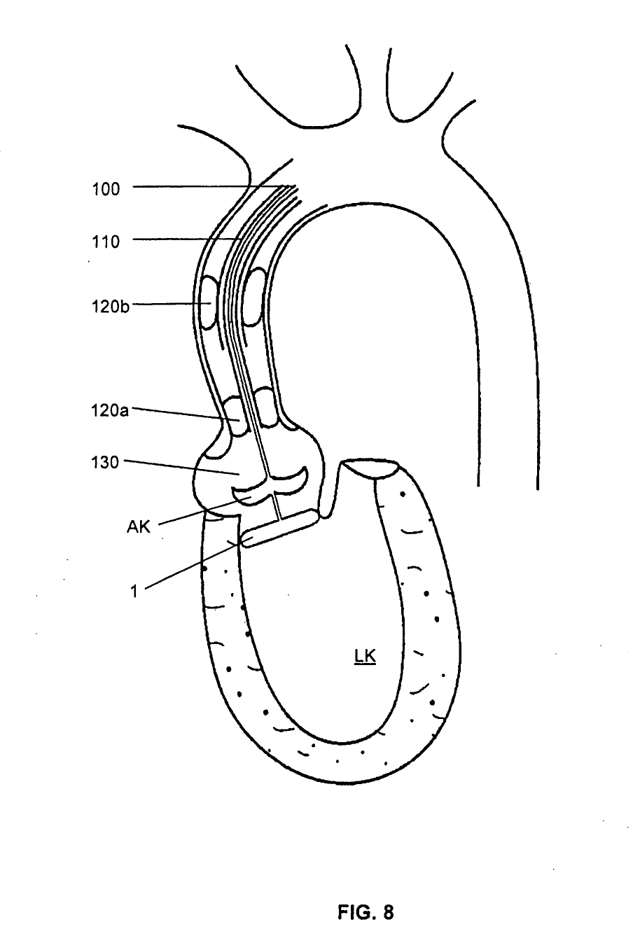

FIG. 8 Cross section of the human heart with the described balloon catheter of the invention for aortic valve ablation in situ

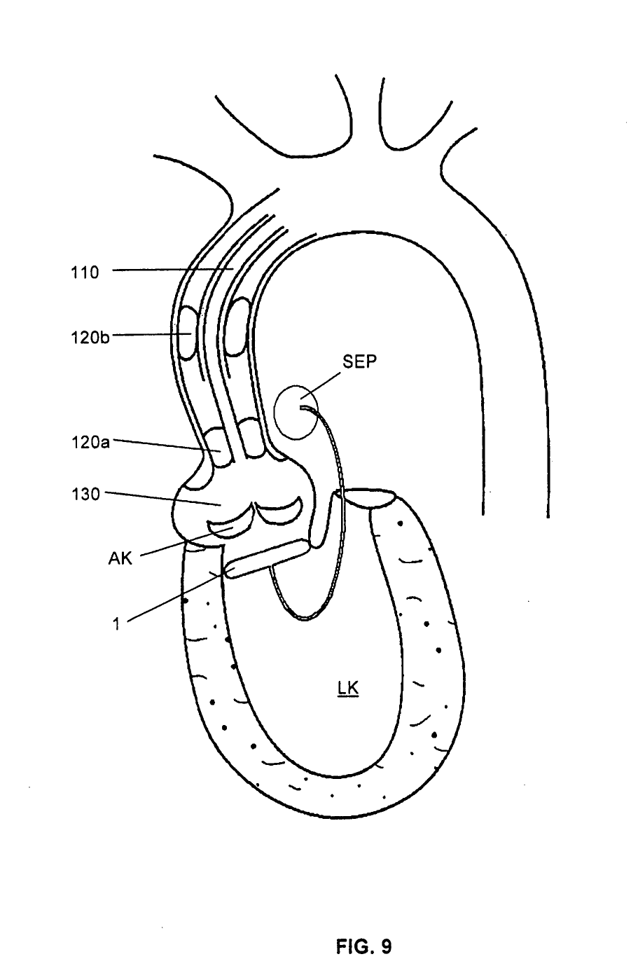

FIG. 9 Cross section of the human heart with another example of the characterized balloon catheter of invention for aortic valve ablation in situ in combination with an additional catheter

FIG. 1 describes a lateral view according to the balloon catheter (1) of the invention. From an external view the balloon catheter of this invention is similar to a conventional catheter with a line part and a balloon part. The special feature of this balloon is a circumferential, preferred discontinuous, limited area 50 which is gas-permeable and generally created of macroscopic pores.

For clarification FIG. 2 indicates the balloon catheter of FIG. 1 from topview as a preferred example. The first line consists of an inner line part 10 and of an additional encircling vacuum line 30. The gas-permeable areas 50 are circularly arranged around the balloon catheter.

FIG. 3 shows a special example of the invention which can be applied also for FIGS. 1 and 2. The balloon catheter consists of a first line 10 which is connected with an inflatable chamber 20. The unit 40 is adjacent to the inflatable chamber 20 forming a cavity with said chamber into which a vacuum line 30 opens. At the outer site of the unit 40 at least one limited area 50 is gas-permeable. The limited area 50 of balloon catheter 1 circulates at the outer site of the unit and is regularly interrupted by gas-permeable pores. FIG. 1 demonstrates a special example in which the vacuum line 30 envelopes the first line 10 and the unit 40 envelopes the chamber 20.

To position the balloon catheter 1 in situ the chamber 20 has to be deflated. To completely inflate the chamber 20, gas or fluids have to be led in via the first line 10. At the time of extension of the chamber 20 the adjacent unit 40 will be also extended. The maximum extension of unit 40 will be reached with maximum extension of chamber 20.

FIG. 4 demonstrates a subsequent example. After achievement of maximal extension of the chamber 20, the gas-permeable areas 50 at the outside of unit 40 form trumpet-like protuberances. These protuberances promote the suction of the balloon catheter to the environmental tissue.

To avoid a collapse of unit 40 due to vacuum, the cavity is stabilized by special separator elements which are resisting this collapse. Preferentially, these separator elements (see FIG. 5) build conduits 60 which will lead to the gas-permeable areas 50 where they preferentially end into pores at the outside (FIG. 4). To establish a constant suction at all gas-permeable areas 50, the supply line system of the separator elements 60 should be reasonably branched out, as has been illustrated in FIG. 6.

FIG. 7 shows a special design of the invention. The separator elements are joined with a connecting element 80. These separator elements 70, as so called circular convexities or protrusions, are filled with gas or fluids to maintain the interspace between the chamber 20 and the unit 40.

FIG. 8 demonstrates a constructed example of a catheter of invention for aortic valve ablation. The already described catheter, DE 102 17 559, has been combined with this new invention. The balloon catheter consists of a perfusion catheter 100, several dilatation units 120a, 120b, 1, and a port channel 110 through which the working tools can be positioned into the working area 130. The working area 130 encloses the aortic valve AK. The dilatation unit 120b supports the guidance of the catheter. The dilatation unit 120a seals the working area 130 to its proximal side. The distal dilatation unit, balloon catheter 1 of the invention, accurately seals the working area 130 to the left heart chamber LK. Another possibility of positioning of the balloon catheter 1 exists and can also maintain the interruption of the bloodstream: it can be placed deeper into the left ventricle LK or into the left atrium. To interrupt the bloodstream, vacuum will be established at the unit 40 through the vacuum line 30 which enables the cavity 40 to be drawn into intimate contact with the left ventricular outflow tract of the left heart chamber and with the mitral valve.

FIG. 9 illustrates a cross section of the human heart in situ with a subsequent designed example of a catheter of invention in combination with another catheter. The well known catheter of DE 102 17 559 exists of labeled elements (FIG. 2) without the distal dilatation unit. This balloon catheter 1 of invention is not connected to the catheter. It can be placed minimally invasive into the left ventricle LK via the septum SEP. The advantage of this construction creates significantly more space for the required ablation tools in the port channel 110 to reach the working area 130 for aortic valve ablation.

In conclusion, the procedural steps for aortic valve replacement with this balloon catheter of invention are characterized as followed:
establishment of the cardiopulmonary bypass in a familiar fashion, ie. in the groin
application of the cardioplegic solution via the ascending aorta or the coronary sinus
insertion and positioning of the distal balloon catheter of invention into the left ventricular outflow tract of the left heart chamber, into the left heart chamber, or into the left atrium. This can be done via the aorta through the heart valve or preferably straight to the left ventricular area via the atrial septum of the heart. To hold the balloon catheter in place, vacuum will be applied to draw it into intimate contact with the left ventricular outflow tract and with the mitral valve.
insertion and positioning of additional occlusion catheters to block the coronary arteries,
insertion and positioning of an additional proximal balloon catheter of invention upside the aortic valve to create an ablation chamber. In the ablation chamber the resection of the heart valve can be easily performed with catheter-guided tools (as water jet, laser, endoscope, suction, grab catheter, etc.).
   The advantage of this procedure is a significantly enlarged lumen of the proximal inserted catheter for aortic valve ablation compared to commercially available catheters. The invented catheter facilitates the placement of a larger amount of tools or other or bigger tools via the cavity into the working area.

1. Balloon catheter (1) for the sealing of blood vessels and cardiac chambers with at least one inflatable chamber (20) connected to a first line (10), characterized by a unit (40) adjacent to the at least one inflatable chamber (20), forming a cavity with said chamber (20) into which a vacuum line (30) opens, whereby separator elements (60,70) are provided between the walls forming the cavity, which is gas permeable at least in a limited region (50) on the outside therefore, for drawing the balloon catheter (1) into intimate contact with the blood vessel or with the cardiac chamber wall.

2. Balloon catheter (1) of claim 1, characterized by a unit (40) which is completely enveloping the inflatable chamber (20).

3. Balloon catheter (1) of one of the preceding claims, characterized by a vacuum line (30) enveloping the first line (10).

4. Balloon catheter (1) of one of the preceding claims, characterized by the separator elements (60,70) which are forming connecting lines from the vacuum line (30) to the at least one gas-permeable limited region (50).

5. Balloon catheter (1), of one of the preceding claims, characterized by a at least one gas-permeable limited region (50) offering at least one pore.

6. Catheter with at least one hollow channel (110) and at least two dilatation units (120a, 1) which are interspaced of each other and connected to the distal end of the long extended catheter, is characterized by at least one of the dilatation units (120a, 1) forming a balloon catheter of one of the claims 1 to 5.
